SEQUENCE LISTING (17 sequences)

SEQ ID NO: 1 - protein, 11 aa, Homo sapiens
  PEPTIDE (1)..(11): heavy Chain Complementary Determining Region
SGFTFNTNAMN

SEQ ID NO: 2 - protein, 19 aa, Homo sapiens
  PEPTIDE (1)..(19): heavy Chain Complementary Determining Region
RIRSKSNNYATYYADSVKD

SEQ ID NO: 3 - protein, 9 aa, Homo sapiens
  PEPTIDE (1)..(9): heavy Chain Complement Determining Region
DRGWGAMDY

SEQ ID NO: 4 - protein, 11 aa, Homo sapiens
  PEPTIDE (1)..(11): light Chaim Complement Determining Region
SASQDINNYLN

SEQ ID NO: 5 - protein, 7 aa, Homo sapiens
  PEPTIDE (1)..(7): light Chaim Complement Determining Region
YTSSLHS

SEQ ID NO: 6 - protein, 9 aa, Homo sapiens
  PEPTIDE (1)..(9): light Chaim Complement Determining Region
QQFSNLPWT

SEQ ID NO: 7 - protein, 214 aa, Mus musculus
  PEPTIDE (1)..(214)
DIQMTQTTSSLSASLGDRVTISCSASQDINNYLNWYQQKPDGTVKLLIYYTSSLHSGVPSRFSGSGSGTDYSLTISNLEPEDFATYYCQQFSNLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 108 aa, Mus musculus
  PEPTIDE (1)..(108)
DIQMTQTTSSLSASLGDRVTISCSASQDINNYLNWYQQKPDGTVKLLIYYTSSLHSGVPSRFSGSGSGTDYSLTISNLEPEDFATYYCQQFSNLPWTFGGGTKLEIKR

SEQ ID NO: 9 - protein, 119 aa, Mus musculus
  PEPTIDE (1)..(119)
EVQLVETGGGLVQPKGSLKLSCAASGFTFNTNAMNWVRQAPGKGLEWVARIRSKSNNYATYYADSVKDRFTLSRDDSQSMLYLQMNNLKTEDTAMYYCVRDRGWGAMDYWGQGTTVTVS

SEQ ID NO: 10 - protein, 119 aa, Mus musculus
  PEPTIDE (1)..(119)
EVQLVESGGGLVQPKGSLKLSCAASGFTFNTNAMNWVRQAPGKGLEWVARIRSKSNNYATYYADSVKDRFTLSRDDSQSMLYLQMNNLKTEDTAMYYCVRDRGWGAMDYWGQGTTVTVS

SEQ ID NO: 11 - protein, 119 aa, Mus musculus
  PEPTIDE (1)..(119)
GVQLVETGGGLVQPKGSLKLSCAASGFTFNTNAMNWVRQAPGKGLEWVARIRSKSNNYATYYADSVKDRFTLSRDDSQSMLYLQMNNLKTEDTAMYYCVRDRGWGAMDYWGQGTTVTVS

SEQ ID NO: 12 - protein, 825 aa, Homo sapiens
  PEPTIDE (1)..(825)
  ref <308>: DATABASE ACCESSION NUMBER: NP_000409.1
  ref <309>: DATABASE ENTRY DATE: 2008-03-16
  ref <313>: RELEVANT RESIDUES IN SEQ ID NO: (1)..(825)
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 13 - protein, 107 aa, Artificial
  Huanized Polypeptide
  PEPTIDE (1)..(107)
DIQMTQSPSSLSASVGDRVTITCSASQDINNYLNWYQQKPGKAPKLLIYYTSSLHSGVPSRFSGSGSGTDFTLTISSLQPEDFATYYCQQFSNLPWTFGGGTKLEIK

SEQ ID NO: 14 - protein, 120 aa, Artificial
  hunaized polypeptide
  PEPTIDE (1)..(120)
EVQLVESGGGLVKPGGSLRLSCAASGFTFNTNAMNWVRQAPGKGLEWVARIRSKSNNYATYYADSVKDRFTLSRDDSKNTLYLQMNSLKTEDTAVYYCTRDRGWGAMDYWGQGTTVTVSS

SEQ ID NO: 15 - protein, 120 aa, Artificial
  humanized polypeptide
  PEPTIDE (1)..(120)
EVQLVESGGGLVKPGGSLRLSCAASGFTFNTNAMNWVRQAPGKGLEWVARIRSKSNNYATYYADSVKDRFTISRDDSKNTLYLQMNSLKTEDTAVYYCTRDRGWGAMDYWGQGTTVTVSS

SEQ ID NO: 16 - protein, 508 aa, Artificial [sequence omitted]

SEQ ID NO: 17 - protein, 513 aa, Artificial [sequence omitted]

(19) United States
(12) Patent Application Publication
(10) Pub. No.: US 2011/0008326 A1
(43) Pub. Date: Jan. 13, 2011

(54) BINDING AGENTS DIRECTED AGAINST IL-4 RECEPTOR FOR THE TREATMENT OF TUMORS, INFLAMMATORY AND IMMUNOLOGICAL DISORDERS

(76) Inventors: Oliver Hill, Neckarsteinach (DE); Marcus Branschaedel, Heidelberg (DE); Christian Gieffers, Dossenheim (DE); Meinolf Thiemann, Schriesheim (DE); Christian Merz, Neckarsteinach (DE)

Correspondence Address: HOWREY LLP-CA, C/O IP DOCKETING DEPARTMENT, 1299 Pennsylvania Avenue, NW, Room B-3, Washington, DC, 20004-2402, US

(21) Appl. No.: 12/922,433

(22) PCT Filed: Mar. 30, 2009
(86) PCT No.: PCT/EP09/53756

§ 371 Date: Sep. 13, 2010

(30) Foreign Application Priority Data
Apr. 2, 2008 (EP) 08006750.7

(51) Int. Cl. A61K 39/395; C07K 16/28; A61P 11/06; A61P 35/00; A61P 37/00; A61P 29/00; A61P 19/02; A61P 31/06; A61P 17/00

(52) U.S. Cl. 424/131.1; 530/389.1; 530/387.3; 530/388.22; 530/387.2; 424/172.1; 424/133.1; 424/143.1; 424/135.1; 424/139.1; 424/136.1

(57) ABSTRACT

The present invention relates to the use of an antigen-binding agent directed against human interleukin-4 receptor for the prevention and/or treatment of tumors, inflammatory and immunological disorders. Further the invention relates to methods of inhibiting the bioactivity of IL-4 without inhibiting binding of IL-4 to IL-4R and particularly to methods for treatment and/or prevention of tumors, inflammatory and immunological disorders, the methods comprising administering to an individual in need thereof an antigen-binding agent with binding affinity for IL-4R.

### BACKGROUND

The present invention relates to the use of an antigen-binding agent thereof directed against human interleukin-4 receptor for the prevention and/or treatment of tumors, inflammatory and immunological disorder.

WO 2004/069274 refers to the use of cytokine antagonists which modulate the expression and/or the function of a cytokine for the down-regulation of an anti-apoptotic protein in a cell. In particular, it is referred to the use of cytokine antagonists for the treatment of cancer. Antibodies directed against cytokine-receptors are indicated as examples of cytokine antagonists.

WO 01/92340 A2 describes use of IL-4 antagonists for treating medical conditions induced by IL-4. In particular, it is referred to the use of IL-4 antagonists for the treatment of inflammatory diseases, including the treatment and/or prevention of allergic conditions and asthma. Antibodies specific for IL-4 receptors are indicated as examples of IL-4 antagonists, wherein the antibodies are characterized by inhibiting IL-4-induced biological activity and IL-13-induced biological activity. The treatment of cancer with such antibodies, however, is neither disclosed nor suggested.

The formation of homo-oligomers or hetero-oligomers appears to be the crucial event during activation and transmembrane signalling of cytokine receptors, IL-4 signalling is mediated through a heterodimeric complex of two cytokine receptor proteins, IL-4Rα and the γ-chain of the IL-2 receptor system, designated γ_{c} (Kondo et al., 1993, Russell et al., 1993). Some cells respond to IL-4 without using γ_{c}, by recruiting an alternative subunit , i. e. a component of the IL-13 receptor complex, into the receptor complex (Aman et al., 1996).

It was surprisingly found that the monoclonal antibody (mAb) X2145 described by Tony et al. (1994), which was raised against interleukin-4Rₑₓ, i. e. the extracellular domain of the interleukin-4 receptor α subunit (IL-4Rα) is useful to inhibit IL-4- and also IL-13-induced responses and is especially suitable for the treatment of tumors, inflammatory and immunological disorders.

Thus, the present invention refers to the use of antibodies or antigen-binding fragments specific for human interleukin-4 receptor for the manufacture of a medicament, particularly for the prevention and/or treatment of tumors, inflammatory and immunological disorders, wherein the antibodies and antibody fragments preferably inhibit both IL-4-induced and IL-13 -induced biological activities.

### BRIEF DESCRIPTION OF THE DRAWINGS

FIG. 1: Amino Acid Sequences of the humanized and mouse variable light and heavy chains.

FIG. 2: Amino Acid Sequences of the CDRs of the anti-IL-4R antibodies (SEQ ID NO: 1-6).

FIG. 3: Amino Acid Sequence of the anti-IL4R-scFv-Fc and anti-IL4-scFv-Fc binding agent constructs.

FIG. 4: Amino Acid Sequence of IL-4R polypeptide NP_{—}000409_version 1 (SEQ ID NO 12) with indication of the domain structure.

FIG. 5: Crystal Structure of IL-4R

FIG. 6: Graph displaying the growth of TF-1 cells in the presence of anti-IL-4R/anti-IL-4 and bi-specific antigen-binding agents

FIG. 7: Graph displaying the results of a metabolic assay of TF-1 cells in the presence of anti-IL-4R antigen binding agents

FIG. 8: Graphs displaying the results of an ELISA experiment using mouse anti-IL-4R antibody and humanized anti-IL-4R antibody

FIG. 9: Models of IL-4-IL-4R interaction to illustrate the beneficial effect of binding of an anti-IL-4R antigen binding agent in a way that does not interfere with the IL-4 binding to the receptor.

### BRIEF DESCRIPTION OF THE INVENTION

In a first embodiment relates to a method for inhibiting the bioactivity of IL-4 comprising administering to an individual in need thereof a bi-specific antigen-binding agent, wherein said antigen-binding agent comprises at least one heavy chain variable region and at least one light chain variable region, wherein the amino acid sequence of the complementarity determining regions (CDRs) of the heavy chains are i)

SGFTFNTNAMN (SEQ ID NO:1), ii) RIRSKSNNYATYYADSVKD (SEQ ID NO:2); iii) DRGWGAMDY (SEQ ID NO:3); and iv) a sequence derived by substituting 1, 2 or 3 amino acids of SEQ ID NOs: 1, 2 and/or 3; and/or the amino acid sequences of the complementarity determining regions (CDRs) of the light chain are: i) SASQDINNYLN (SEQ ID NO:4); ii) YTSSLHS (SEQ ID NO:5); iii) QQFSNLPWT (SEQ ID NO:6); and iv) a sequence derived by substituting 1, 2 or 3 amino acids of SEQ ID NOs: 4, 5, and/or 6, and wherein said bi-specific antigen-binding agent comprises one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc. . .

The invention further pertains to the following embodiments:

A method for inhibiting the bioactivity of IL-4 comprising administering to an individual in need thereof a bi-specific antigen-binding agent with binding affinity for IL-4R and one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . .

A method for inhibiting the bioactivity of IL-4 comprising administering to an individual in need thereof a bi-specific antigen-binding agent with binding affinity for IL-4R and one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . . , wherein the IL-4R binding of the antigen-binding agent does not interfere with the binding of IL-4 to IL-4R.

A method for inhibiting the bioactivity of IL-4 comprising administering to an individual in need thereof a bi-pecific antigen-binding agent directed against an epitope naturally present on IL-4R comprising one or more amino acids located within a region selected from the group comprising amino acids H87-L89, R173-Y175 or T178-P182, D102-A125, W104-A125, W111-A125, H120, W111, K112, K116, T211, Y179 and R185 of SEQ ID NO: 12 and one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . .

A method for inhibiting the bioactivity of IL-4 comprising administering to an individual in need thereof an antigen-binding agent directed against IL-4R, wherein said antigen-binding agent does not interfere with the binding of IL-4 to IL-4R.

A method for inhibiting the bioactivity of IL-4 comprising administering to an individual in need thereof an antigen-binding agent directed against IL-4R, wherein said inhibits the IL-4 bioactivity and wherein the antigen-binding agent does not interfere with the binding of IL-4 to IL-4R.

A method for inhibiting the bioactivity of IL-4 comprising administering to an individual in need thereof an antigen-binding agent directed against an epitope naturally present on IL-4R comprising one or more amino acids located within a region selected from the group comprising amino acids H87-L89, R173-Y175 or T178-P182, D102-A125, W104-A125, W111-A125, H120, W111, K112, K116, T211, Y179 and R185 of SEQ ID NO: 12.

A method for inhibiting the bioactivity of IL-4 comprising administering to an individual in need thereof an antigen-binding agent, wherein said antigen-binding agent comprises at least one heavy chain variable region and at least one light chain variable region, wherein the amino acid sequence of the complementarity determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and iv) a sequence derived by substituting 1, 2 or 3 amino acids of SEQ ID NOs: 1, 2 and/or 3; and/or the amino acid sequences of the complementarity determining regions (CDRs) of the light chain are: i) SASQDINNYLN (SEQ ID NO:4); ii) YTSSLHS (SEQ ID NO:5); iii) QQFSNLPWT (SEQ ID NO:6); and iv) a sequence derived by substituting 1, 2 or 3 amino acids of SEQ ID NOs: 4, 5, and/or 6.

A method for treatment of cancer, inflammatory and immunological disorders comprising administering to an individual in need thereof a bi-specific antigen-binding agent, wherein said antigen-binding agent comprises at least one heavy chain variable region and at least one light chain variable region, wherein the amino acid sequence of the complementarity determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and iv) a sequence derived by substituting 1, 2 or 3 amino acids of SEQ ID NOs: 1, 2 and/or 3; and/or the amino acid sequences of the complementarity determining regions (CDRs) of the light chain are: i) SASQDINNYLN (SEQ ID NO:4); ii) YTSSLHS (SEQ ID NO:5); iii) QQFSNLPWT (SEQ ID NO:6); and iv) a sequence derived by substituting 1, 2 or 3 amino acids of SEQ ID NOs: 4, 5, and/or 6, and wherein said bi-specific antigen-binding agent comprises one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . .

A method for treatment of cancer, inflammatory and immunological disorders comprising administering to an individual in need thereof a bi-specific antigen-binding agent with binding affinity for IL-4R and one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . .

A method for treatment of cancer, inflammatory and immunological disorders comprising administering to an individual in need thereof a bi-specific antigen-binding agent with binding affinity for IL-4R and one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . . , wherein the IL-4R binding of the antigen-binding agent does not interfere with the binding of IL-4 to IL-4R.

A method for treatment of cancer, inflammatory and immunological disorders comprising administering to an individual in need thereof a bi-specific antigen-binding agent directed against an epitope naturally present on IL-4R comprising one or more amino acids located within a region selected from the group comprising amino acids H87-L89, R173-Y175 or T178-P182, D102-A125, W104-A125, W111-A125, H120, W111, K112, K116, T211, Y179 and R185 of SEQ ID NO: 12 and one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . .

A method for treatment of cancer, inflammatory and immunological disorders comprising administering to an individual in need thereof an antigen-binding agent directed against IL-4R, wherein said antigen-binding agent does not interfere with the binding of IL-4 to IL-4R.

A method for treatment of cancer, inflammatory and immunological disorders comprising administering to an individual in need thereof an antigen-binding agent directed against IL-4R, wherein said inhibits the IL-4 bioactivity and wherein the antigen-binding agent does not interfere with the binding of IL-4 to IL-4R.

A method for treatment of cancer, inflammatory and immunological disorders comprising administering to an individual in need thereof an antigen-binding agent directed against an epitope naturally present on IL-4R comprising one or more amino acids located within a region selected from the group comprising amino acids H87-L89, R173-Y175 or T178-P182, D102-A125, W104-A125, W111-A125, H120, W111, K112, K116, T211, Y179 and R185 of SEQ ID NO: 12.

A method for treatment of cancer, inflammatory and immunological disorders comprising administering to an individual in need thereof an antigen-binding agent, wherein said antigen-binding agent comprises at least one heavy chain variable region and at least one light chain variable region, wherein the amino acid sequence of the complementarity determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and iv) a sequence derived by substituting 1, 2 or 3 amino acids of SEQ ID NOs: 1, 2 and/or 3; and/or the amino acid sequences of the complementarity determining regions (CDRs) of the light chain are: i) SASQDINNYLN (SEQ ID NO:4); ii) YTSSLHS (SEQ ID NO:5); iii) QQFSNLPWT (SEQ ID NO:6); and iv) a sequence derived by substituting 1, 2 or 3 amino acids of SEQ ID NOs: 4, 5, and/or 6.

An antigen-binding agent directed against IL-4R that does not interfere with the binding of IL-4 to IL-4R for treatment of cancer, inflammatory and immunological disorders.

An antigen-binding agent directed against IL-4R that inhibits the IL-4 bioactivity and does not interfere with the binding of IL-4 to IL-4R for treatment of cancer, inflammatory and immunological disorders.

An antigen-binding agent directed against an epitope naturally present on IL-4R comprising one or more amino acids located within a region selected from the group comprising amino acids H87-L89, R173-Y175 or T178-P182, D102-A125, W104-A125, W111-A125, H120, W111, K112, K116, T211, Y179 and R185 of SEQ ID NO: 12 for treatment of cancer, inflammatory and immunological disorders.

An antigen-binding agent, wherein said antigen-binding agent comprises at least one heavy chain variable region and at least one light chain variable region, wherein the amino acid sequence of the complementarity determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and iv) a sequence derived by substituting 1, 2 or 3 amino acids of SEQ ID NOs: 1, 2 and/or 3; and/or the amino acid sequences of the complementarity determining regions (CDRs) of the light chain are: i) SASQDINNYLN (SEQ ID NO:4); ii) YTSSLHS (SEQ ID NO:5); iii) QQFSNLPWT (SEQ ID NO:6); and iv) a sequence derived by substituting 1, 2 or 3 amino acids of SEQ ID NOs: 4, 5, and/or 6, for treatment of cancer, inflammatory and immunological disorders.

A bi-specific antigen-binding agent with binding affinity for IL-4R and one further binding affinity, wherein said antigen-binding agent comprises at least one heavy chain variable region and at least one light chain variable region, wherein the amino acid sequence of the complementarity determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and iv) a sequence derived by substituting 1, 2 or 3 amino acids of SEQ ID NOs: 1, 2 and/or 3; and/or the amino acid sequences of the complementarity determining regions (CDRs) of the light chain are: i) SASQDINNYLN (SEQ ID NO:4); ii) YTSSLHS (SEQ ID NO:5); iii) QQFSNLPWT (SEQ ID NO:6); and iv) a sequence derived by substituting 1, 2 or 3 amino acids of SEQ ID NOs: 4, 5, and/or 6, and wherein said bi-specific antigen-binding agent comprises one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc., for treatment of cancer, inflammatory and immunological disorders.

A bi-specific antigen-binding agent with binding affinity for IL-4R one further binding affinity, the further binding affinity being e.g, for a cytokine molecule or a cytokine receptor molecule such as e.g. IL-4, IL-13, IL-5, IL-6; IL-10; IL-10R; IL-13R, common gamma-chain, CXCR4, etc., for treatment of cancer, inflammatory and immunological disorders.

A bi-specific antigen-binding agent with binding affinity for IL-4R one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc . . . , wherein the IL-4R binding of the antigen-binding agent does not interfere with the binding of IL-4 to IL-4R, for treatment of cancer, inflammatory and immunological disorders.

A bi-specific antigen-binding agent directed against an epitope naturally present on IL-4R comprising one or more amino acids located within a region selected from the group comprising amino acids H87-L89, R173-Y175 or T178-P182, D102-A125, W104-A125, W111-A125, H120, W111, K112, K116, T211, Y179 and R185 of SEQ ID NO: 12 and one further binding affinity, the further binding affinity being e.g. for a cytokine molecule or a cytokine receptor molecule such as e.g. IL-4, IL-13, IL-5, IL-6; IL-10; IL-10R; IL-13R, common gamma-chain, CXCR4, etc., for treatment of cancer, inflammatory and immunological disorders.

In yet further embodiments the invention relates to the use of the antigen-binding agents and/or the bi-specific antigen-binding agents as described above for the manufacture of medicaments for treatment of tumors, inflammatory and immunological disorders.

### DETAILED DESCRIPTION OF THE INVENTION

IL-4 induces specific biological functions in a wide range a cells and is one of the major regulatory cytokines of the immune system. IL-4 serves as an autocrine growth factor and is a signature cytokine of TH2 cells. Simultaneously, IL-4 inhibits the development of the TH1 cell subset and antagonises the IFN-gamma-mediated activation of genes, thus being an important mediator of the TH2 immune response. Misregulation of the TH2 response can lead to allergic reactions like asthma (Vercelli, 2006). Furthermore, IL4 has a stimulatory effect on proliferation and differentiation of activated B cells. In proliferating B cells, it induces antibody isotype switching to IgE, IgG2 and IgG4. On monocytes and macrophages, 14 up-regulates the expression of MHC class-Il molecules and soluble cytokine inhibitors (Kindt et al., 2006),

Though IL-4 and its corresponding receptor were supposed to be predominatly expressed on cells of the immune system and some non-haematopoietic cells, it has been shown that tumour cells from a large variety of origins express the IL-4R and/or IL4. Moreover, IL4 was shown to protect cancer cells from chemotherapy-induced apoptosis, and antagonists of IL4-signalling caused down-regulation of anti-apoptotic proteins and a re-sensitisation of tumours toward chemotherapy-induced apoptosis (Todaro et al., 2007, 2008).

The substances and methods of the invention may used for treatment and prevention of IL-4-induced conditions. Such conditions include conditions caused or exacerbated, directly or indirectly, by IL-4. Also other factors or cytokines may play a role in the said conditions, but IL-4 induces or mediates the condition at least in part. As the biological activities of IL-4 are mediated through binding to interfeukin-4 receptor (IL-4R). IL-4-induced conditions include those arising from biological responses that result from the binding of IL-4 to a native IL-4 receptor on a cell, or which may be inhibited or suppressed by preventing IL-4 from binding to an IL-4 receptor. Conditions that may be treated include, but are not limited to, medical disorders characterized by abnormal or excess expression of IL-4, or by an abnormal host response to IL-4 production. Further examples are conditions in which IL-4-induced antibody production, or proliferation or influx of a particular cell type, plays a role. IL-4-induced disorders include those in which IL-4 induces upregulation of IL-4 receptors or enhanced production of another protein that plays a role in a disease (e. g., another cytokine). In preferred embodiments the disorder are particularly tumors and inflammatory and immunological disorders.

The term “tumor” or “tumors” in all it's grammatical forms as used in the context of the present invention may comprise tumors of the head and the neck, tumors of the respiratory tract, tumors of the anogenital tract, tumors of the gastrointestinal tract, tumors of the urinary system, tumors of the reproductive system, tumors of the endocrine system, tumors of the central and peripheral nervous system, tumors of the skin and its appendages, tumors of the soft tissues and bones, tumors of the lymphopoietic and hematopoietic system, etc. Tumors may comprise for example neoplasms such as benign and malignant tumors, cancer, carcinomas, sarcomas, leukemias, lymphomas or dysplasias.

Cancer comprises any malignant neoplasm or spontaneous growth or proliferation of cells. In certain embodiments of the invention cancer comprises invasive cancer. A subject having cancer, for example, may have a leukemia, lymphoma, or other malignancy of blood cells. In certain embodiments refers to a solid tumor.

In a particular embodiment, the tumor is for example cancer of the head and the neck, cancer of the respiratory tract, cancer of the anogenital tract, cancer of the gastrointestinal tract, cancer of the skin and its appendages, cancer of the central and peripheral nervous system, cancer of the urinary system, cancer of the reproductive system, cancer of the endocrine system, cancer of the soft tissues and bone, cancer of the hematopoietic and lymphopoietic system. Exemplary solid tumors include but are not limited to colon tumor, colon tumor, a cervical tumor, a gastric tumor, and a pancreatic tumor, non small cell lung cancer (NSCLC), testicular cancer, lung cancer, ovarian cancer, uterine cancer, cervical cancer, pancreatic cancer, colorectal cancer (CRC), breast cancer, as well as prostate, gastric, skin, stomach, esophageal, and bladder cancer.

Inflammatory and immunological disorders may be imflammatory disorders of the head and the neck, the respiratory tract, the anogenital tract, the gastrointestinal tract, the urinary system, the reproductive system, the endocrine system, the central and peripheral nervous system, the skin and its appendages, the soft tissues and bones, the lymphopoietic and hematopoietic system, etc.

Examples of inflammatory and immunological disorders include, but are not limited to, systemic lupus erythematosus (SLE), discoid lupus, lupus nephritis, sarcoidosis, inflammatory arthritis, including juvenile arthritis, rheumatoid arthritis, psoriatic arthritis, Reiter's syndrome, inflammation of the heart (myocarditis), inflammation of the kidneys (nephritis), bursitis, tendonitis, Hodgkins's disease, chronic obstructive pulmonary disease (“COPD”), thyroiditis, rheumatic fever, myasthenia gravis, Behcet's syndrome, sarcoidosis, polymyositis, conjunctivitis, gingivitis, periarteritis nodosa and aplastic anemia ankylosing spondylitis, and gouty arthritis, rejection of an organ or tissue transplant, hyperacute, acute, or chronic rejection and/or graft versus host disease, multiple sclerosis, hyper IgE syndrome, polyarteritis nodosa, primary biliary cirrhosis, inflammatory bowel disease, Crohn's disease, celiac's disease (gluten-sensitive enteropathy), autoimmune hepatitis, pernicious anemia, autoimmune hemolytic anemia, psoriasis, scleroderma, myasthenia gravis, autoimmune thrombocytopenic purpura, autoimmune thyroiditis, Grave's disease, Hasimoto's thyroiditis, immune complex disease, chronic fatigue immune dysfunction syndrome (CFIDS), polymyositis and dermatomyositis, cryoglobulinemia, thrombolysis, cardiomyopathy, pemphigus vulgaris, pulmonary interstitial fibrosis, Type I and Type II diabetes mellitus, type 1, 2, 3, and 4 delayed-type hypersensitivity, allergy or allergic disorders, unwanted/unintended immune responses to therapeutic proteins, asthma, Churg-Strauss syndrome (allergic granulomatosis), atopic dermatitis, allergic and irritant contact dermatitis, urtecaria, IgE-mediated allergy, atherosclerosis, vasculitis, idiopathic inflammatory myopathies, hemolytic disease, Alzheimer's disease, chronic inflammatory demyelinating polyneuropathy, and the like. In some other embodiments inflammatory disorders may include for pulmonary inflammation, including, but not limited to, lung graft rejection, asthma, sarcoidosis, emphysema, cystic fibrosis, idiopathic pulmonary fibrosis, chronic bronchitis, allergic rhinitis and allergic diseases of the lung such as hypersensitivity pneumonitis, eosinophilic pneumonia, bronchiolitis obliterans due to bone marrow and/or lung transplantation or other causes, graft atherosclerosis/graft phlebosclerosis, as well as pulmonary fibrosis resulting from collagen, vascular, and autoimmune diseases such as rheumatoid arthritis and lupus erythematosus.

Inflammatory disorders may in certain embodiments comprise chronic inflammatory disorders defined as a disease process associated with long-term activation of inflammatory cells (leukocytes). The chronic inflammation may lead to damage of patient organs or tissues.

Inflammatory and immunological disorders may be imflammatory disorders of the head and the neck, the respiratory tract, the anogenital tract, the gastrointestinal tract, the urinary system, the reproductive system, the endocrine system, the central and peripheral nervous system, the skin and its appendages, the soft tissues and bones, the lymphopoietic and hematopoietic system, etc.

In certain embodiments the inflammatory disease may be a infections disease or a disease caused by parasites. Such diseases comprise e.g. tuberculosis, infections by enterobacteria, infections by mycopalsma etc.

In one especially preferred embodiment the inflammatory disorder is asthma.

The term “antigen-binding agent” as used in the context of the present invention shall refer to antibodies, antibody fragments, antigen-binding fragments of antibodies, mini-antibodies and other molecules specifically binding to antigens. In certain embodiments the antigen-binding agents shall e.g. be whole antibodies, Fabs, F(ab′)2 fragments, Fd fragments, disulfide-linked Fvs (scFvs), anti-idiotypic (anti-Id) antibodies, and scFvs, single chain antibodies, miniantibodies, fragments of antibodies such as e.g. Fab' fragments, affibodies (or affybodies), trinectins, monobodies, FN3 monobodies, anticalins or suitable antibody mimetics. The antibodies or antibody fragments may include one or more of the components or domains found in whole antibodies comprising e.g. the heavy chain (CDR-H), the variable domain (V) of the complementarity determining region (CDR) of a heavy chain (CDR-H, VH) and a light chain (CDR-L, VL). The antibodies, antibody fragments and antigen-binding fragments of the present invention may be polyclonal or monoclonal. The antibodies, antibody fragments and antigen-binding fragments of the invention may be derived form any species comprising but not limited to mouse, rat, dog, cat, sheep, goat, rabbit, hamster, opossum, humans, horse, apes, primates, cow, shark or whale. Further antibodies may comprise also genetically engineered antibodies and/or antibodies generated in transgenic animals, microorganisms, plants or antibodies generated synthetically. In certain embodiments the antibodies may comprise human or humanized antibodies. Likewise the binding agents used herein may be humanized to minimize the risk of any immune-response in human beings. Generally any molecule with specific binding affinity to a specified antigen may be used as an antigen-binding agent of the invention.

In certain embodiments the antigen-binding agent is a chimeric or humanized antibody which has human constant domains, e.g. human constant IgG1, IgG2, IgG3 or IgG4 domains. Further, a fully human antibody is preferred which may be manufactured by phage display techniques or in transgenic animals having a human immune system. More preferably, the antibody is a humanized or human antibody which additionally comprises human or substantially human framework regions. Also preferred are antibody fragments, e.g. divalent or monovalent antibody fragments such as F(ab)2 fragments. On the other hand, the antibody may be a recombinant antibody, e.g. a single chain antibody or a fragment thereof, e.g. an scFv fragment.

In one embodiment the antibody of the invention is an antibody or an antibody fragment, e.g. a chimeric or humanized antibody derived from the murine antibody X2/45 (Tony et al., 1994) produced by the hybridoma cell line DSM ACC2882. The hybridoma cell line DSM ACC2882 was deposited under the Budapest Treaty for the Deposit of Microorganisms on January 29, 2008 at Deutsche Sammlung von Mikroorganismen and Zellkulturen GmbH (DSMZ), Mascheroder Weg 1b, 38124 Braunschweig, Germany.

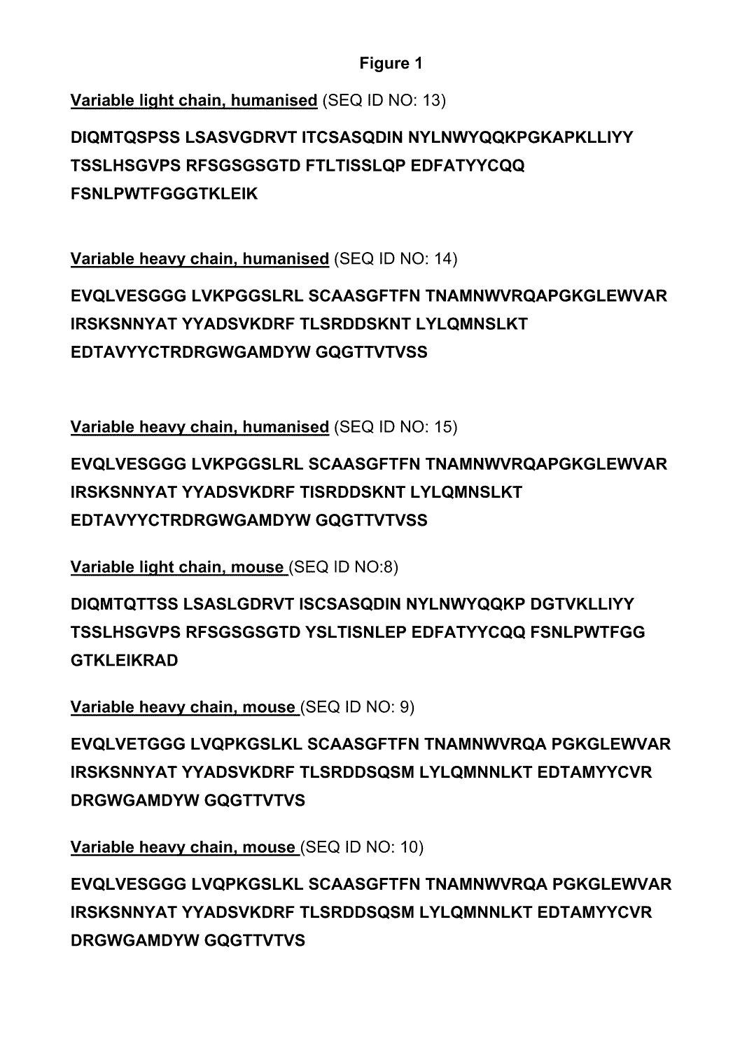

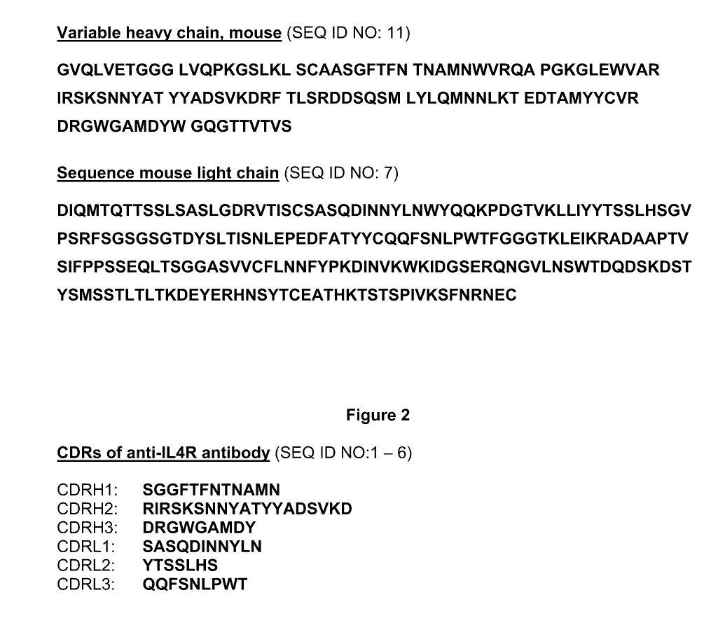

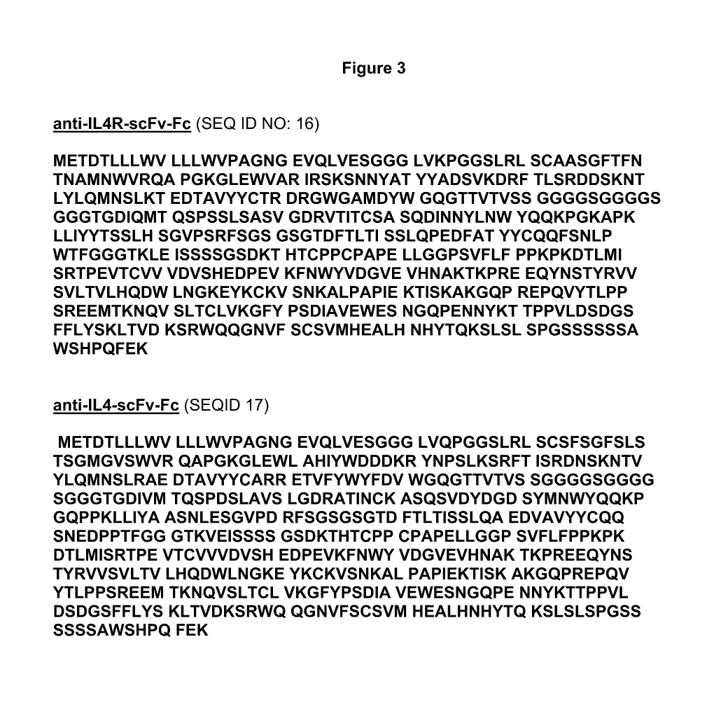

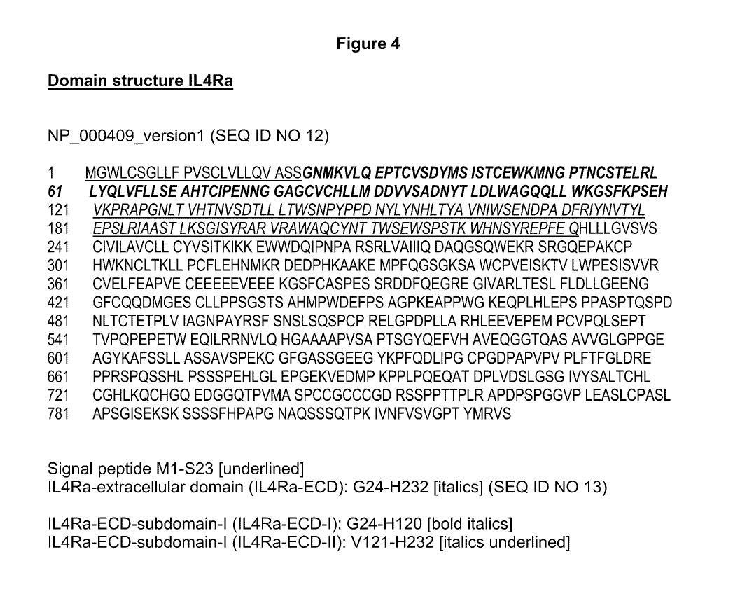

The murine antibody X2/45 comprises the light chain amino acid sequence of SEQ ID NO:7 or the variable region thereof (SEQ ID NO: 8) (FIG. 1) and a heavy chain amino acid sequence selected from VH1 (SEQ ID NO:9), VH2 (SEQ ID NO:10) and VH3 (SEQ ID NO:11) (FIG. 2).

A further preferred embodiment relates to the use of an antibody directed against the IL-4 receptor, such as 6-2, 12B5, 63, 1B7, 5A1, and 27A1 as disclosed in WO 01/92340 A2 (the content of which is herein incorporated by reference), or an antibody or an antibody fragment derived therefrom, e.g. a chimeric or humanized antibody. This chimeric or humanized antibody preferably comprises the complementarity determining regions of the heavy and/or light chain of any of the antibodies 6-2, 12B5, 63, 1B7, 5A1, and 27A1. It is particularly referred to the amino acid sequences of the light chain and heavy chain variable region disclosed in WO 01/92340 A2.

Further, the invention refers to an antibody that recognizes the same epitope region of human IL-4 receptor as an antibody selected from 6-2, 12B5, 63, 1 B7, 5A1, and 27A1, or an antigen-binding fragment thereof, for the manufacture of a medicament for the prevention and/or treatment of cancer.

IL-4R or IL-4 receptor as used herein shall refer to the interleukin 4 receptor alpha chain isoform a polypeptide with the sequence of SEQ ID NO: 12 which is also found under the accession number NP_{—}000409.1. The polypeptide sequence provided under this accession number Version 1 shall be incorporated herein by reference. Any references to amino acid positions on the IL-4R protein as made herein refer to the amino acid sequence given in SEQ ID NO 12.

Generally the antigen-binding agent of the invention is directed against any antigenic stretch of amino acids of the sequence of IL-4R that is located in the extracellular domain of the polypeptide, which is located at G24-H232 of SEQ-ID NO 12.

In certain embodiments the antigen-binding agent is directed against an epitope of the IL-4R polypeptide in a way, that binding of the antigen-binding agent to the epitope does neither interfere with the interaction of IL-4R and IL-4 polypeptides nor with binding of IL-4 to IL-4R polypeptide. Generally this can be accomplished for any epitopes being located on the three dimensional structure of the IL-4R protein on the side opposite to the location of the IL-4 binding region. Further also epitopes being located on the same side of the protein as the IL-4 binding region may not interfere with the IL-4R IL-4 interaction given that the respective epitope is located in a way that the antigen-binding agent when bound to the epitope does not sterically hinder IL-4 protein from accessing and binding to binding region on IL-4R.

In certain embodiments the antigen-binding agent is an antigen-binding agent specific for human interleukin-4 receptor, wherein said antigen-binding agent comprises at least one heavy chain variable region and at least one light chain variable region, wherein the amino acid sequence of the complementarity determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and iv) a sequence derived by substituting 1, 2 or 3 amino acids of SEQ ID NOs: 1, 2 and/or 3; and/or the amino acid sequences of the complementarity determining regions (CDRs) of the light chain are: i) SASQDINNYLN (SEQ ID NO:4); ii) YTSSLHS (SEQ ID NO:5); iii) QQFSNLPWT (SEQ ID NO:6); and iv) a sequence derived by substituting 1, 2 or 3 amino acids of SEQ ID NOs: 4, 5, and/or 6, for treatment of cancer, inflammatory and immunological disorders.

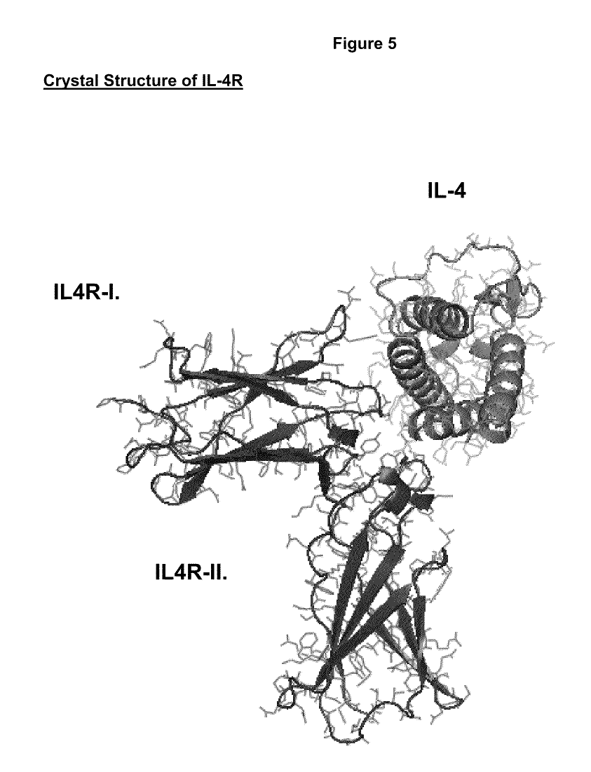

For the human IL-4R several crystal structures of the extracellular domain have been published. The human IL-4R-ECD consists of two subdomains both bearing an Fibronectin-III like fold. (cf. FIG. 5) The N-terminal subdomain (IL-4R-ECD-I, referred to as IL4R-I. in the drawing), as derived from the structural data, comprises amino acids Gly23 to His120, the C-terminal subdomain (IL-4R-ECD-II, referred to as IL4R-II. in the drawing) comprises amino-acids Val121 to His232. The IL4-binding pocket is localized inbetween the subdomains and residues from both subdomains are involved in ligand binding. In certain embodiments the binding agent is directed against an epitope positioned in the interdomain connecting sequence.

In certain embodiments the epitopes are localized in a way, that the binding agent of such epitopes does not impair the binding of IL-4 to the IL-4R-ECD, but impairs the association of IL-13R or of the Common gamma-chain to the IL-4R-chain thereby inhibiting the IL-4 and IL-13 signalling cascade.

The term “epitope” as used herein shall refer to an antigenic determinant that interacts with a specific antigen binding site on an antigen-binding agent. In certain embodiments the epitope is an antigenic determinant recognized by the variable region of an antigen binding agent such as e.g. paratopes, namely natural, synthetic or artificial paratopes. Also, epitopes can be defined as antigenic determinants interacting with proteins specifically designed and selected for this binding purpose, e.g. anticalins.

A single antigene may have more than one epitope. Epitopes may be either linear or conformational. A conformational epitope is one produced by spatially juxtaposed amino acids from different segments of the linear polypeptide chain and a linear epitope is produced by adjacent amino acid residues in a polypeptide chain. In certain circumstances, an epitope may comprise residues of mono-, oligo-, or poly-saccharides, phosphoryl groups, or sufonyl groups on the antigen.

In certain embodiments the antigen-binding agent is directed against a linear epitope on the sequence of the IL-4R (SEQ ID NO 12). In a preferred embodiment the epitope is naturally present on the IL-4R polypeptide. In certain embodiments the linear epitope has a length of 3, 4, 5, 6, 7, 8, 9, 10, 11, 12, 13, 14, 15, 16, 17, 18 19 or 20 amino acids. In certain embodiments the length of the epitope may be 1-20, 2-18 or 4-15 amino acids. In a preferred embodiment the length of an epitope has a length of 3-10 or 3-6 amino acids.

The preferred antigen-binding agent is directed against an epitope that is naturally occurring on IL-4R and is located on SEQ ID NO 12. In a certain embodiments the antigen-binding agent is directed against an epitope positioned on SEQ ID NO 12 namely, within any one of the regions defined by amino acids D102-A125, W104-A125, W111-A125. In one embodiment the antigen-binding agent is directed against an epitope comprising at least W104, Q107 and Q108. In another embodiment, the epitope comprises at least one of the amino acids W111, K112, K116, H120 and T211, or comprises one ore more of the amino acids within any one of the regions defined by amino acids H87-L89, R173-Y175 or T178-P182, D102-A125, W104-A125, W111-A125, H120, W111, K112, K116, T211, Y179 and R185 of SEQ ID NO: 12.

In one embodiment the antigen-binding agent may be directed against an epitope that may be conformational or linear and is naturally occurring on the IL-4R that comprises one or more of the amino acids W111, K112, K116, H12, W111, K112, K116, H120 and T211, Y179 and R185 of SEQ ID NO: 12.

In another embodiment, the antigen-binding agent is derected against an epitope comprising at least the amino acids W111, K112, K116, H120 of SEQ ID NO: 12. In another embodiment the epitope comprises one ore more of residues T178-P182 of SEQ ID NO: 12 and additionally residue R185. In yet another embodiment the epitope comprises at least residues Y179 and R185 of SEQ ID NO: 12. In certain embodiments conformational epitopes may comprise amino acids acids i) W111, K112, K116 or ii) H12 or W111, K112, K116, H120 and T211.

The antibody may be a complete antibody, e.g. an IgG antibody, or an antigen-binding fragment thereof.

In a further embodiment of the present invention, the antibody comprises a further different specific binding component. For example, the antibody or antibody fragment may be a fusion polypeptide with the further component or a bi-specific antibody. The antibody may recognize in addition to the human IL-4 receptor also another antigen, e.g. a further cytokine which is associated with cancer, e. g. IL-4 or IL-10, wherein it is preferred that the further binding component is specific for IL-4.

In a preferred embodiment of the invention, the antigen-binding agent is bi-specific. Bi-specific as used herein shall mean that the antigen-binding agent is specific for and binding affinity for two different epitopes. Such epitopes may be located on the same or on different molecules. In certain preferred embodiments of the invention the bi-specific antigen-binding agent is a bi-specific antibody or bi-specific fragment of an antibody.

According to the invention the bi-specific antigen-binding agent may have specificities for two different cytokine and/or cytokine receptor molecules. In certain embodiments such cytokines and/or cytokine receptor molecules are e.g. IL-4R, IL-4, ILS, IL6; 110; IL-13, IL1OR; IL-13R, common gamma-chain or CXCR4. In one preferred embodiment the bi-specific antigen-binding agent has a first specificity for IL-4R polypeptide and one further specificity for e.g. IL-4, IL5, IL6; IL10; IL-13, IL1OR; IL-13R, common gamma-chain or CXCR. In one preferred embodiment the binding of the bi-specific antigen-binding agent to IL-4R does neither interfere with the interaction of IL-4R and IL-4 polypeptides nor with binding of IL-4 to IL-4R polypeptide.

In certain preferred embodiments the bi-specific antigen-binding agent is a binding agent recognizing both IL-4 and IL-4 receptor, wherein the region specific for the IL-4 receptor may or may not recognize the IL-4 binding site of said receptor. Further, binding of the bi-specific antibody to the IL-4 receptor may or may not inhibit binding of IL-4 to the receptor.

In an especially preferred embodiment, the bi-specific antibody comprises
(a) at least one heavy chain variable region and at least one light chain variable region specific for IL-4 receptor, wherein the amino acid sequence of the complementarity determining regions are as described above; or
(b) at least one heavy chain variable region and at least one light chain variable region which recognizes the same epitope on IL-4 receptor as the region of (a);and
(c) at least one heavy chain variable region and at least one light chain variable region specific for IL-4, wherein the amino acid sequence of the complementarity determining regions are preferably as described in WO2007/107349 (which incorporated herein by reference in its entirety); or
(d) at least one heavy chain variable region and at least one light chain variable region which recognizes the same epitope on IL-4 as the region of (c).

The cytokine IL-4 and its receptor IL-4R have been shown to play an important role in the pathogenesis of allergy-related illnesses. Furthermore, it has been reported that different types of cancer cells are protected from chemotherapy-induced apoptosis in an autocrine manner by expression of IL-4. Inhibition of the IL-4/ILR system is therefore a promising therapeutic tool in the treatment of both allergies and tumours.

Without wishing to be bound by theory, applicant assumes that, when the above described bi-specific antibody recognizing both IL-4 and the IL-4 receptor binds to the IL-4 receptor, IL-4, which has already bound to the IL-4 receptor or which will bind to the IL-4 receptor, will be ‘trapped’. As a consequence of binding of the bi-specific antibody to the IL-4 receptor, γ_{c} or a component of the IL-13 receptor complex, i. e. neither IL-13 receptor α which has bound IL-13 nor IL-13 receptor α alone, will be recruited into the receptor complex, thus efficiently blocking IL-4 and IL-13 mediated signalling.

Applicant further assumes that, at the same time, IL-13-induced responses mediated by IL-13 binding to IL-13 receptor and recruitment of IL-4 receptor to this complex is efficiently inhibited.

It is a further advantage of said bi-specific antibody that no crosslinking of IL-4 receptors occurs, thus allowing for an amended dose window.

The inventors surprisingly found that an antigen binding agent directed against IL-4R antibody inhibiting the bioactivity of IL-4 that does not interfere with the interaction between IL-4R and IL-4 has an improved effect on inhibition of IL-4 bioactivity compared to binding agents that are directed against IL-4R that do interfere with the receptor ligand interaction. Especially in in-vivo models it could be shown that reduction of tumor growth is more strongly inhibited by binding agents that do not interfere with the binding of IL-4 to the IL-4 receptor than by those that do not allow the binding of IL-4 to the receptor. The inventors found that the inhibition of the IL-4 bioactivity for such binding agents is based on the ability to interfere with interactions of the IL-4R with other molecules involved in the IL-4 cascade such as e.g. with IL-13 or the common gamma chain. Moreover it is known that tumor cells may autocrinely or paracrinley produce IL-4. The improved effect of an antigen binding agent according to the invention is based on the effect that IL-4 may despite binding of the antigen binding agent to the IL-4R still be captured by the IL-4R and thereby is removed from the environment and prevented from exhibiting it's bioactivity to IL-R receptor molecules that have not been bound by the antigen binding agent. Accordingly there is a potentiating effect of the inhibition of the IL-4 bioactivity as the signal transduction is interrupted and in addition the blocked receptor acts as an antagonist of IL-4 in binding IL-4 without effecting the bioactivity. The method according to the invention transforms the functional IL-4 Receptor to an nonfunctional IL-4 antagonist in-vivo and thereby not only blocks the receptor but also antagonizes available IL-4 in the cellular environment.

Based on this mechanism the inventors found that the effect may even be increased by adding further binding specificities to the antibody that additionally act as cytokine antagonists either for IL-4 or other cytokines such as e.g. IL-4, IL-13, IL-5, IL-6; IL-10; IL-10R; IL-13R, common gamma-chain, CXCR4, etc . . .

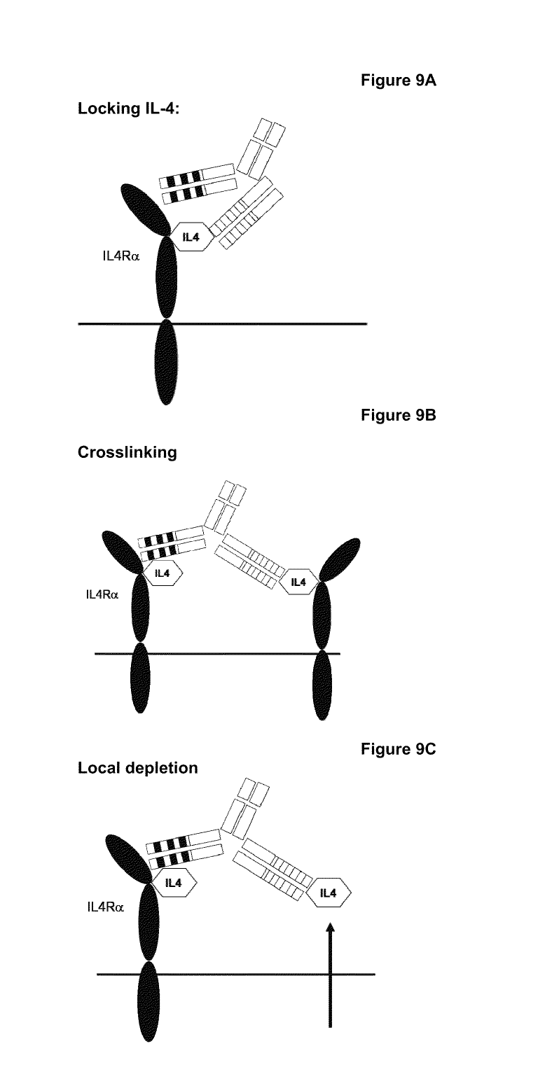

In certain embodiments the bi-functional antibody with one binding site specific for IL-4 and one binding site specific for IL-4R will have the technical advantage that such bi-specific antibody has increased therapeutic effect. Inhibition of the IL-4 bioactivity is effected in such antibody not only by binding to the IL-4 receptor but also by additional interactions. Details are given in FIG. 9, Possible advantageous of a bi specific antibody are for example the locking of receptor-bound IL-4 by interaction with IL-4, in the receptor bound state and additional binding to the identical IL-4R chain. Cf. FIG. 9 A), cross-linking of receptors in an IL-4-dependent manner, by binding to IL-4 in the receptor bound state and additional binding to a second IL-4R (cf. FIG. 9 B) and depletion of autocrine IL-4 on IL-4R-bearing cells (cf. FIG. 9 C). In the case. of cancer it has been described that tumor cells express IL-4 and the respective receptor(s) necessary for IL-4 dependent signalling, in particular IL-4R. Binding of a bi-specific antibody to the IL-4R on an IL-4 expressing cell would therefore deplete IL-4 immediately after secretion from the cell and prevent IL-4 dependent signalling.

Multispecific antigen-binding agents and antibodies capable of binding two or more antigens are well-known in the art. There is a variety of methods available for the preparation of said antibodies, such as cell fusion, chemical conjunction or recombinant DNA techniques, Preferred methods suitable for the production of multispecific antibodies are described in WO2007/024715 A2, the content of which is herein incorporated by reference.

Specifically, for the production of a bi-specific antibody a binding protein according to WO2007/024715 A2 is used, comprising a polypeptide chain, wherein said polypeptide chain comprises VDI-(X1)n-VD2-C-(X2)n, wherein VD1 is a first variable domain, VD2 is a second variable domain, C is a constant domain, X1 represents an amino acid or polypeptide, X2 represents an Fc region an n is 0 or 1. It is especially preferred, that VD1 and VD2 in the binding protein are heavy chain variable domains. More preferably, the heavy chain variable domain is selected from a group consisting of a human heavy chain variable domain, a CDR grafted heavy chain variable domain, and a humanized heavy chain variable domain. It is preferred, that VD1 and VD2 are capable of binding different antigens.

The specificity and the kinetics of a bi-specific antigen-binding agent binding to IL-4 and the IL-4 receptor may be determined in-vitro by ELISA. Therefore, different concentrations of the IL-4 receptor may be immobilized and the affinity of the bi-specific antibodies may be measured in the presence or absence of IL-4. Preferably, the IL-4 receptor is used as a recombinant protein in monomeric form at sufficiently low concentrations at which receptor crosslinking is substantially excluded. Samples without antibody may be used as a control.

The dissociation constant (off-rate) and association constant (on-rate) of the bi-specific antigen-binding agents may be determined using the BlAcore or Quartz Crystal Microbalance systems. The efficiency of the bi-specific antibodies may be further tested in a TF-1 proliferation bioassay (R&D Systems).

In a still further embodiment, the anti-IL-4 receptor antigen-binding agent may be used in combination with a further separate cytokine antagonist, e. g. an antibody which is specific for a cancer-associated cytokine such as IL-4 or IL-10, an antagonistic cytokine mutein, e. g. an antagonistic IL-4 mutein or a soluble cytokine receptor.

The anti-IL-4 receptor antigen-binding agent is preferably administered parenterally, e.g. by injection or infusion. For this purpose, the antibody is formulated as a pharmaceutical composition in a physiologically acceptable carrier, optionally together with physiologically acceptable excipients. The weekly dose is preferably in the range of 0.1 mg/kg to 10 mg/kg, more preferably 1 mg/kg to 5 mg/kg, most preferably about 2 mg/kg. The administration is carried out for a time period sufficient to obtain the desired beneficial effect, e.g. induction of a tumor response to treatment. The antibody therapy should then be maintained for a predetermined period, e.g. several weeks.

The antigen-binding agent is preferably administered in combination with further anti-tumor therapy, e.g. radiation therapy and/or with at least one further medicament, e.g. a chemotherapeutic agent, a cytokine antagonist, a death signal pathway activator, and/or an anti-tumor antibody. In an especially preferred embodiment, the anti IL-4 receptor antibody is administered in combination with radiation therapy and/or at least one chemotherapeutic agent. In a further especially preferred embodiment, the anti IL-4 receptor antibody is administered together with a further cytokine antagonist antibody, e.g. an anti IL-4 antibody, an anti IL-10 antigen-binding agent or an antagonistic IL-4 mutein such as the R121D/R124D IL-4 mutein, in combination with radiation therapy and/or at least one chemotherapeutic agent.

The combination therapy may be administered throughout the whole treatment or an interval thereof. For example, the treatment may comprise a first interval wherein the anti IL-4 receptor antibody, optionally together with a further anti-cytokine antibody, is administered without radiation therapy and/or chemotherapy alone and subsequent intervals wherein (i) the IL-4 receptor antibody, optionally together with a further anti-cytokine antibody, is administered with radiation therapy and/or further medicaments, e.g. chemotherapy and/or (ii) radiation therapy and/or further medicaments are administered without the anti IL-4 receptor antibody.

Alternatively, a first treatment interval may comprise combined therapy and a subsequent treatment interval may comprise single therapy, i.e. radiation therapy and/or administration of further medicaments without the anti IL-4 receptor antigen-binding agent, optionally alternating with combined therapy.

In particular, death pathway activators may be selected from TRAIL or TRAIL muteins (Kelley et al., 2005; MacFarlane et al., 2005; Van der Sloot et al., 2006), DR4 ligand or DR5 ligand and muteins thereof. Further, agonistic antigen-binding agent against death receptors, such as TRAIL-R, DR4 or DR5 are suitable.

In particular, chemotherapeutic agents which may be used in combination with the monoclonal antibodies of the present invention preferably are antineoplastic compounds. Such compounds included in the present invention comprise, but are not restricted to (i) antimetabolites, such as cytarabine, fludarabine, 5-fluoro-2′-deoxyuridine, gemcitabine, hydroxyurea or methotrexate; (ii) DNA-fragmenting agents, such as bleomycin, (iii) DNA-crosslinking agents, such as chlorambucil, platinum compounds, e.g. cisplatin or oxaliplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphteria toxin; (vi) topoisomerase I inhibitors, such as camptothecin or topotecan; (vii) topoisomerase II inhibitors, such as etoposide (VP-16) or teniposide; (viii) microtubule-directed agents, such as colcemide, colchicine, taxanes, e.g. paclitaxel, vinblastine or vincristine; (ix) kinase inhibitors such as flavopiridol, staurosporine or derivatives thereof, e.g. STI571 (CPG 57148B) or UCN-01 (7-hydroxystaurosporine); (x) miscellaneous agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; or antibiotics, such as doxycyclin; (xi) hormones such as glucocorticoids or fenretinide; (xii) hormone antagonists, such as tamoxifen, finasteride or LHRH antagonists.

In an especially preferred embodiment of the present invention, the chemotherapeutic agent is selected from the group consisting of platinum compounds, e.g. cisplatin or oxaliplatin, doxorubicin and taxanes, e.g. paclitaxel or etoposide.

Generally the antigen-binding agents of the invention may be sued for the treatment of tumors, e.g. tumors of the head and the neck, tumors of the respiratory tract, tumors of the anogenital tract, tumors of the gastrointestinal tract, tumors of the urinary system, tumors of the reproductive system, tumors of the endocrine system, tumors of the central and peripheral nervous system, tumors of the skin and its appendages, tumors of the soft tissues and bones, tumors of the lymphopoietic and hematopoietic system, etc. Tumors may comprise for example neoplasms such as benign and malignant tumors, cancer, carcinomas, sarcomas, leukemias, lymphomas or dysplasias.

Cancers that may be treated with the antigen binding agents according to the invention comprise any malignant neoplasm or spontaneous growth or proliferation of cells. In certain embodiments of the invention cancer comprises invasive cancer. A subject having cancer, for example, may have a leukemia, lymphoma, or other malignancy of blood cells. In certain embodiments refers to a solid tumor.

In a particular embodiment, the tumor is for example cancer of the head and the neck, cancer of the respiratory tract, cancer of the anogenital tract, cancer of the gastrointestinal tract, cancer of the skin and its appendages, cancer of the central and peripheral nervous system, cancer of the urinary system, cancer of the reproductive system, cancer of the endocrine system, cancer of the soft tissues and bone, cancer of the hematopoietic and lymphopoietic system. Exemplary solid tumors include but are not limited to colon tumor, colon tumor, a cervical tumor, a gastric tumor, and a pancreatic tumor, non small cell lung cancer (NSCLC), testicular cancer, lung cancer, ovarian cancer, uterine cancer, cervical cancer, pancreatic cancer, colorectal cancer (CRC), breast cancer, as well as prostate, gastric, skin, stomach, esophageal, and bladder cancer.

The antigen-binding agents of the invention may also be used for treatment of inflammatory and immunological disorders that are associated with IL-4 bioactivity. Examples of such inflammatory and immunological disorders are given above in this text.

Particularly, the antigen-binding agents can be used for the treatment of cancer types which are associated with increased IL-4 and/or IL-13 expression and/or which are at least partially resistant to apoptosis due to the expression of anti-apoptotic proteins.

Examples of such cancer types comprise neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, bladder carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidal melanoma, seminoma, rhabdomyosarcoma, craniopharyngeoma, osteosarcoma, chondrosarcoma, myosarcoma, liposarcoma, fibrosarcoma, Ewing sarcoma and plasmocytoma. Further, the antibodies may be used for the treatment of Minimal Residual Disease (MRD).

In a particularly preferred embodiment, the IL-4 receptor antigen-binding agents according to the present invention can be used for the treatment of non-lymphoid and non-myeloid cancers, more preferably epithelial cancers, particularly solid tumors.

Especially preferred examples of cancer types where the use of the IL-4 receptor antigen-binding agents according to the present invention is particularly advantageous include all forms of thyroid carcinomas (medullary thyroid carcinoma, papillary thyroid carcinoma, follicular thyroid carcinoma, anaplastic thyroid carcinoma), breast carcinoma, lung carcinoma, prostate carcinoma, colon carcinoma, bladder carcinoma, gastric carcinoma, liver carcinoma, kidney carcinoma, glioblastome, and MRD. Most preferably, the IL-4 receptor antibodies are useful for the treatment of colon or pancreas carcinoma, preferably in combination with further therapy as described above. For the treatment of colon carcinoma, IL-4 receptor antibodies are preferably administered together with chemotherapy and/or radiation therapy. For the treatment of thyroid carcinoma, IL-4 receptor antibodies are preferably administered together with IL-4 antibodies, IL-10 antibodies and together with chemotherapy and/or radiation therapy.

In an especially preferred embodiment, the IL-4 receptor antigen-binding agents according to the present invention are used for the treatment of bladder carcinoma. In a typical treatment scheme, the tumor is removed by surgery if it has not invaded the muscle tissue. Concomitantly, BCG (Bacillus Calmette-Guérin) may be administered as an immunostimulatory agent, either systemically or directly into the bladder.

If the tumor has invaded the surrounding tissue (muscle tissue, adipose tissue), usually the affected area of the urinary bladder will be removed (partial or radical cystectomy). Administration of a chemotherapeutic agent optionally in combination with an antigen-binding agent of the invention at the time of surgery considerably improves the survival rate. It is especially preferred to administer a combination of the chemotherapeutic agents cisplatin, methotrexate and vinblastine before, during and/or after surgery.

For the treatment of late-stage human bladder carcinoma (HBC) chemotherapy is used, optionally in combination with radiation therapy and/or the IL-4 receptor antibodies according to the present invention.

As chemotherapeutic agent useful for administration in late-stage HBC gemcitabine is administered, optionally in combination with paclitaxel, cisplatin, carboplatin and/or methotrexate.

In another especially preferred embodiment, the IL-4 receptor antigen-binding agents according to the present invention are used for the treatment of pancreas carcinoma. Typically, a combination therapy of chemotherapeutic agents, including gemcitabine, optionally in combination with erlotinib, 5-FU and/or taxotere and the monoclonal antibodies of the present invention is applied.

In an further especially preferred embodiment, the IL-4 receptor antigen-binding agents according to the present invention are used for the treatment of colon carcinoma. Following surgical removal of the tumor, if possible, preferably FOLFOX combination (5-FU or capecitabine, leukovorin, oxaliplatin) is administered as adjuvant chemotherapy. Late-stages of the disease characterized by metastasis are preferably treated by bevacizumab in addition to either FOLFOX or FOLFIRI (5-FU, leucovorin and irinotecan). Alternatively or additionally, cetuximab may be used together with irinotecan, however, in the latter case, it is required to determine the patient's EGFR-status prior to chemotherapy.

In another especially preferred embodiment, the IL-4 receptor antigen-binding agents according to the present invention are used for the treatment of gastric carcinoma. Typically, the tumor is removed surgically, accompanied by palliative chemotherapy characterized by the administration of a chemotherpeutic agent selected from 5-FU, BCNU and methyl-CCNU, doxorubicin, mitomycin C or combinations thereof. Further, cisplatin and docetaxel are applied in various combinations. Palliative chemotherapy is preferably combined with administration of the IL-4 receptor antigen-binding agents as supportive therapy.

For low-incidence gastrointestinal stromal tumors (GIST) the highest response rates are obtained using imatinib as chemotherapeutic agent. Concomitant treatment using antibodies according to the invention is highly preferred.

In another especially preferred embodiment, the IL-4 receptor antigen-binding agents according to the present invention are used for the treatment of non-small-cell-lung-cancer (NSCLC) which constitutes the most frequent type of lung carcinoma. In a typical treatment scheme, combination therapy of surgery and radiation therapy is applied, optionally concomitant with IL-4 receptor antigen-binding agents administration. Chemotherapy is applied as palliative treatment and includes administration of cisplatin/carboplatin optionally in combination with bevacizumab. Further adjuvant postoperative treatment regimens include docetaxel and EGFR antagonists such as gefitinib and erlotinib. It is preferred to maintain administration of the IL-4 receptor antigen-binding agents throughout the course of radiation and chemotherapy, optionally as interval treatment.

In another especially preferred embodiment, the IL-4 receptor antigen-binding agents according to the present invention are used for the treatment of head and neck cancer. Treatment is surgical resection of the primary tumor optionally in combination with radiation therapy further optionally accompanied by administration of the antibodies of the invention. Chemotherapy is applied as additional supportive treatment and includes combined administration of paclitaxel and carboplatin. Alternatively, cetuximab is used concomitant with radiation therapy and, more preferably, amifostine, exhibiting cytoprotective effects during radiation therapy. The IL-4 receptor antibodies according to the present invention are preferably used as adjuvant postoperative treatment regimens in combination with chemotherapy.

In an especially preferred embodiment, the IL-4 receptor antibodies according to the present invention are used for the treatment of breast cancer. A typical treatment scheme provides for surgical resection of the primary tumor combined with adjuvant therapy such as radiation, chemotherapy or hormone therapy, including aromatase inhibitors, depending on the type of tumor.

Among the most preferred chemotherapeutic agents are AC or EC, FAC or FEC (A=adriamycin (doxorubicin), C=cyclophosphamide, E=epirubicin, F=fluorouracil). If the lymph knodes are affected additional administration of taxanes is highly preferred. Metastatic breast cancer and breast cancer which is non-responsive to anthracyclines and/or taxane is treated by capecitabine, optionally in combination with recently developed taxane-analogous substances (e. g. epothilone).

Hormone therapy is used for cancers sensitive to hormones with tamoxifen being especially preferred.

Following determination of the HER2/neu status, optionally trastuzumab is used, on its own or in conjunction with chemotherapy or hormone therapy. Trastuzumab can be used as an adjuvant therapy or to treat advanced disease.

Similarly, the IL-4 receptor antigen-binding agents according to the present invention are suitable for adjuvant therapy in early stage as well as in late stage disease, preferably, IL-4 receptor antigen-binding agent administration commences as soon as breast cancer is diagnosed, concomitant with other treatment options or as interval therapy.

The antigen-binding agents of the present invention are especially useful as supportive therapy and are either administered throughout the whole treatment or an interval thereof.

Furthermore, the IL-4 receptor antigen-binding agents according to the present invention are suitable for inducing death of cancer stem cells, e.g. colon cancer stem cells or cancer stem cells in other cancer types as described above. Thus, the antigen-binding agents can be used for the treatment of minimal residual disease (MRD) and/or tumor metastasis. The antigen-binding agents are preferably administered in combination with further therapy as described above.

The disclosure of all patent and non-patent documents recited in the specification above is hereby incorporated by reference in its entirety.

### EXAMPLE 1

### Expression and Purification of Recombinant Binding Agents Directed Against IL-4R

The mouse anti-human IL-4R antibody X2/45 (variable regions shown in SEQID 8, 9, 10, 11) was produced by cultivating the X2/45 hybridoma in PFHM-ll medium (Gibco, Cat. 12040), Cell supernatant was collected and the secreted antibody was affinity purified by protein-A chromatography, followed by size exclusion chromatography (SEC) using a Superdex 200 column (GE Healthcare) with PBS (Invitrogen, Cat. 10001) as running buffer at a flow rate of 0.5 ml/min. The 150 kDa fraction was collected, sterile filtered 0.22 μm and stored below 0° C.

For recombinant protein production, Hek 293T cells grown in DMEM +GlutaMAX (GibCo) supplemented with 10% FBS, 100 units/ml Penicillin and 100 μg/ml Streptomycin were transiently co-transfected with plasmids encoding said proteins. The full length heavy chains of the humanised antibodies (examples for variable regions shown in SEQ ID NO: 14, 15) contained at the C-terminus of the constant regions the Streptag II sequence for purification and detection purposes. The full length humanised light chain (example for the variable region shown in SEQ ID NO: 13) was of kappa type, but lambda may also be applicable. Cell culture supernatant containing recombinant proteins were harvested three days post transfection and clarified by centrifugation at 300 xg followed by filtration through a 0.22 μm sterile filter. For purification of recombinant antibodies with a single specificity, 4 ml of 50% Streptactin Sepharose (IBA GmbH, Gottingen, Germany) were packed to a 2 ml column and equilibrated with 30 ml phosphate buffered saline, pH7.4 (PBS; Invitrogen Cat, 10010). The cell culture supernatant was applied to the column at 4° C. with a flow rate of 2 ml/min. Subsequently, the column was washed with PBS and specifically bound proteins were eluted stepwise by addition of 5×2 ml buffer E (PBS with 2.5 mM Desthiobiotin, pH 7.4). The protein content of the eluate fractions was analysed by absorption scpectroscopy and by silver-stained SDS-PAGE. Postitive fractions were subsequently concentrated by ultrafiltration (Sartorius, Vivaspin, 10,000 Da cut-off) and further analysed by size exclusion chromatography (SEC).

SEC was performed on a Superdex 200 column using an Akta chromatography system (GE-Healthcare). The column was equilibrated with PBS (Invitrogen Cat. 10010) and the concentrated, streptactin purified proteins were loaded onto the SEC column at a flow rate of 0.5 ml/min. The elution of was monitored by absorbance at 280 nm. The apparent molecular weight of purified proteins was determined based on calibration of the Superdex 200 column with gel filtration standard proteins (Bio-Rad GmbH, Munchen, Germany).

For the generation of antibody constructs with two binding specificities, Hek 293T cells were co-transfected as described above and two scFv-FC constructs were used with specificities for either IL-4-(SEQ ID NO: 17) or IL-4R (SEQ ID NO: 16). All scFv-FC constructs were extented C-terminally with the Streptag II sequence. For purification, cleared supernatants were purified on Streptactin Sepharose as described. The Streptactin purified scFv-Fc constructs contained an antibody mixture with the following specificities: a) monospecific IL4 scFv-FC: b) monospecific IL4R-alpha scFv-FC: c) bi-specific scFv-Fc, with one paratop against IL4 and one paratop against IL4R-alpha.

For affinity purification of bi-specific scFv-FC the Streptactin purified mixture of the scFv-FC was sequentially purified on two affinity columns containing immobilised recombinant Interleukin4 and recombinant IL4R-alpha, respectively. The different affinity purified fractions,-IL4-specific, II4R-alpha-specific,- bi-specific, were subsequently analysed with respect to their specificity to recognize their respective antigens by ELISA. In addition all purified antibody fractions were analysed in a cell based proliferation assay for their ability to compete with IL4 induced proliferation on TF1 cells.

### Characterization of the Binding Agents by ELISA Testing

Equal amounts of affinity purified ScFv-FC antibodies were analysed by ELISA for their reactivity towards their respective antigens. The mixture of the Streptactin purified ScFv-FC antibodies showed a predominant reactivity towards IL4, however the affinity purified fractions showed predominantly a specific reaction towards IL4 or IL4R, respectively. The fraction containing putative bi-specific antibodies showed an almost equal reactivity towards both antigens, indicating the bi-specific nature of the purified antibodies.

### EXAMPLE 2

Inhibition of IL-4 induced Proliferation of TF-1 Cells by the Recombinant Binding Agents Proliferation Measured Using a Metabolic Assay

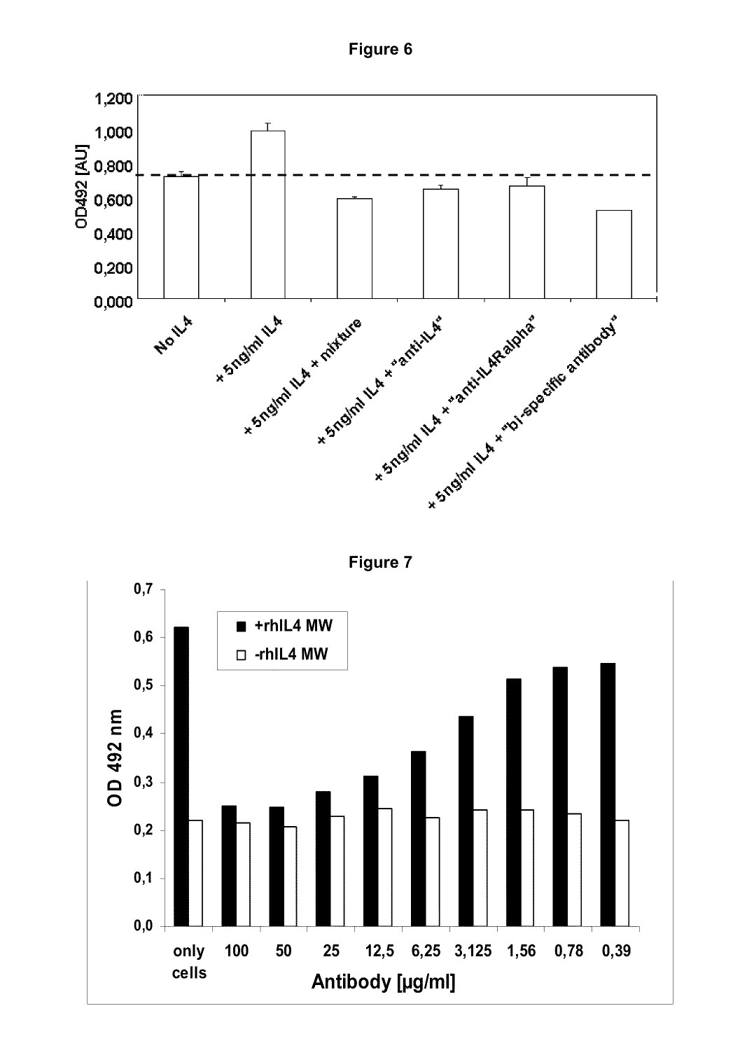

The erythroleukemic human cell line TF-1 was cultured in RPMI-based TF-1 medium supplemented with 2 ng/ml GM-CSF (granulocyte-macrophage colony-stimulating factor). To determine the bioactivity of said proteins, TF-1 cells were harvested by centrifugation, washed with TF-1 medium without GM-CSF and seeded in triplicates at 10,000 cells per well into 96-well plates using TF-1 medium supplemented with human recombinant IL-4 (5 ng/ml) or IL-13 (20 ng/ml) in the presence or absence of said proteins to be analysed. Cells were incubated for three days at 37° C., 5% CO2 and 95% relative humidity. The interleukin-induced proliferation of cells was visualized by a metabolic assay (staining with the tetrazolium compound MTS (Promega)) followed by determination of the absorption at 492 nm. For the competition of IL-4 induced proliferation different fractions of purified ScFv-Fc antibodies (SEQ ID NOS 16, 17) were added at a concentration of 5 μg/ml, as indicated. The resutls are shown in FIG. 6. The dashed line in FIG. 6 shows the proliferation of TF-1 cells without addition of recombinant IL-4 (basis level). Addition of 5 ng/ml IL4 increases the proliferation rate above the basis level. However, addition of purified fractions of ScFv-Fc antibodies competes with the IL-4-induced proliferation. In this experimental setup the mono- and bi-specific antibodies show a comparable activity.

Alternatively, TF-1 cells were co-incubated for three days with varying concentrations of the mouse anti-IL4R antibody X2/45 together with or without 5 ng/ml of recombinant human IL-4 (rhIL4). After three days, cell proliferation was quantified by MTS assay. The results are given in FIG. 7. The addition of IL-4 induces a proliferation rate with an OD 492 nm of approximately 0.6, whereas the background signal of about 0.2 units. The addition of the antibody X2/45 (filled bars) reduces the proliferation of TF-1 cells to the background level (clear bars) in a concentration dependent manner of the X2/45 antibody. Similar results were obtained with humanised versions of X2/45 (for example variable regions are given in SEQ ID NOs: 13, 14, 15)

### EXAMPLE 4

### Generation and Characterization of Humanised Binding Agents Directed Against IL-4R

### Generation of Recombinant Humanised Binding Agents on the Basis of Mouse Binding Agents

The variable domains of the heavy and light chains of the anti-IL-4R specific antibody produced by the mouse hybridoma X2/45 were identified by molecular biology methods. Briefly, total mRNA was isolated and transformed into cDNA using polymerase chain reaction with antibody specific oligonucleotide primers. The resulting fragments were separated by agarose gel elektrophoresis, extracted and cloned into a sequencing vector (TOPO, Invitrogen). Resulting sequences that were encoding for antibody variable regions were used to define the CDRs (Kabat et al., 1991) and the CDRs were subsequently transferred into framework regions of human origin.

Competition Assay for Mouse and Humanised anti-IL-4R Binding Agents

Human, recombinant IL-4R-Fc protein was immobilized on ELISA plates at 100 ng/well followed by blocking of free binding sites. Varying concentrations of the mouse anti-human IL-4R antibody X2/45 (variable regions are given in SEQID 8, 9, 10, 11) was allowed to bind to immobilized receptor for one hour, followed by detection with an anti-mouse specific peroxidase (POD)-conjugated serum. As expected, with increasing concentrations of the mouse antibody, an increasing ELISA signal could be detected, indicating specific binding.

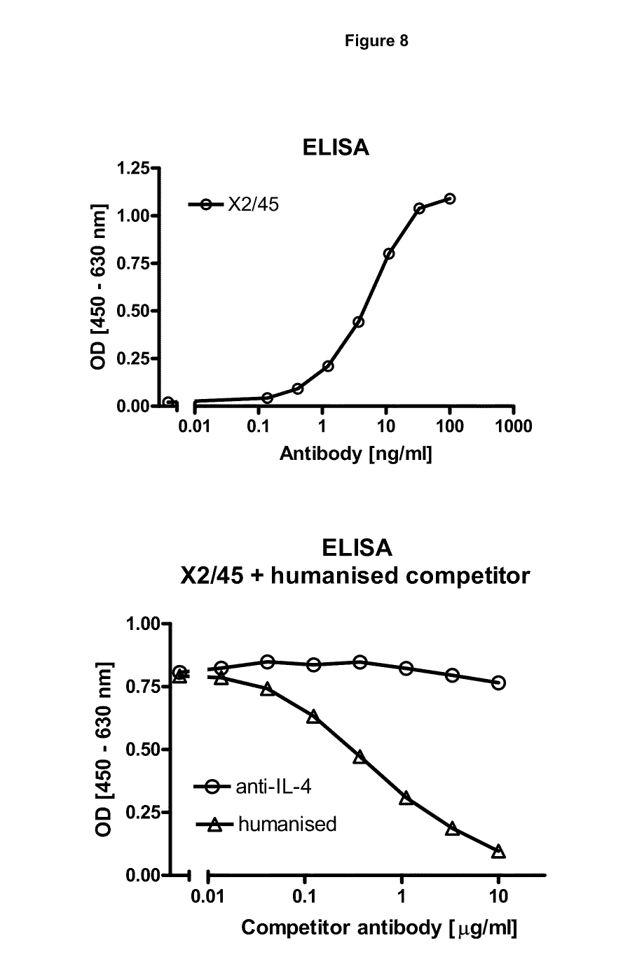

To show that the humanised antibody binds to the same epitope as the parental mouse antibody, ten ng/ml of mouse antibody X2/45 were co-incubated on IL-4R-Fc coated ELISA plates together with varying concentrations of either humanised antibody (variable regions are given in SEQ ID NOs: 13 and 14) or an IL-4-specific control antibody. The mouse mAb was detected with an anti-mouse-POD conjugated serum. The ELISA signal decreased with increasing concentrations of the humanised antibody, indicating that the mouse and humanised IL-4R-specific antibodies recognize the same binding site(s), whereas the control anti-IL-4 antibody had no effect. Results are shown in FIG. 8.

### EXAMPLE 4

Testing of Effect of the IL-4R Binding Agent on Tumor Growth in a in-vivo Model

The efficacy of the IL-4R antibody is shown in an in vivo model using mouse Xenograft tumors. For this experiments IL-4R-positve tumor cells derived from chemotherapy resistant pancreas tumors (e.g: ASPC-1, CAPAN-1, MIA PaCa-2, COLO-357, T3M4, PANC-1 (Prokopchuk, 2005)) or colon tumors (Co10205, HT29), are inocculated subcutanously to immune-compromised mice. Mice showing developing Xenograft tumors are devided into eight treatment groups:
1) buffer control
2) chemotherapy
3) IL-4R antibody competing for IL4 binding
4) IL-4R antibody non competing for IL4 binding
5) Bi-specific antibody (e.g. anti IL-4R/anti IL4)
6) Combination: IL-4R antibody competing+chemotherapy
7) Combination: IL-4R antibody non competing+chemotherapy
8) Combination: Bi-specific-antibody+chemotherapy

Mice are treated for three weeks. A typical treatment schedule incldes one weekly application of IL4R-antibody at a dose of 10 mg/kg/body weight for three weeks. The chemotherapeutic treatment schedule depends on the agent used (e.g.: 5 FU [5 applications/week for 2 weeks], Oxaliplatin [one application/week for three weeks]) The effect on tumor growth is measured by determinig the tumor volume of the respective treatment group.

Treatment with buffer alone (group 1) is not inhibiting growth of Xenograft tumors. Mice treated with chemotherapy only (group2) show a small delay in tumor growth but the final tumor volumes are comparable to the control group. Mice treated intravenously with IL-4R antibodies (group 3,4) or bi-specific antibodies (group 5), respectively, show a delay of tumor growth. However, mice treated with chemotherapy+IL-4R antibodies (group 6, 7) or bi-specific antibodies (group 8), respectively, show a clear reduction of tumor growth and in the best case a total regression of inocculated Xenograft tumors.

A comparison of the different antibody formats reveals that the non competing IL-4R antibody and the bi-specific antibody formats alone show the same or even better tumor reduction activity as the IL-4R antibody that competes with ILA binding, when used in combination with chemotherapy. The combination of the non competing IL-4R antibody and the bi-specific antibody formats with chemotherapy far outreaches the tumor reduction activity that may be achieved by a IL-4R antibody that competes with IL4 binding , when used in combination with chemotherapy.

The result of the Xenograft experiment support the hypothesis that interference of IL4-signalling by IL-4R antibodies or bi-specific antibodies (antibodies that have one specific binding side against IL-4R) is an effective approach for the treatment of solid tumors. Furthermore the hypothesis that an antigen binding agent that inhibits the IL-4 signal transduction pathway but nonetheless does not interfere with the interaction between IL-4 and IL-4 receptor is strongly supported by the experiment. The delay of tumor growth of the IL-4R antibody treated in group3 reveals a single agent activity for the IL4R-antibody. Combination of chemotherapy with IL-4R treatment (group 6,7, 8) indicates a potent synergistic effect on tumor growth.

### EXAMPLE 5

### Testing of Effect of the IL-4R Binding Agents on Tumor Cell Lines

The effect of an antagonistic agent binding to the IL-4R can be tested with the IL-4R positive lung carcinoma cancer cell line A-549 (DSMZ no, ACC 107) or human ductal brast carcinoma cell line BT-549 (ATCC HTB-122), respectively.

Equal numbers of cells are plated to 96 well plates and incubated either with:
1. Buffer control
2. Chemotherapeutic
3. IL-4R binding agent
4. IL-4R binding agent+chemotherapeutic agent

In this experiments an IL-4R specific antibody, a single chain antibody, a FN3 monobody and an anticalin are used as IL-4R binding agents can be.

Cells of the respective treatment groups are grown for 3 days. Subsequently the proliferation rate of the cells is quantified using a metabolic assay (staining with the tetrazolium compound MTS (Promega)) followed by determination of the absorption at 492 nm. In this experimental setup cells of treatment group 1 (Buffer control) and treatment group 2 (chemotherapy) do show rapid proliferation indicated by a high OD492. Cells treated with IL-4R binding agents (group 3) show slightly reduced proliferation rate. However, cells of treatment group 4 (IL-4R-binding+chemo) show a significant reduction of proliferation. The reduction rate of cellular proliferation in group 4 (and partially also group3) is seen independent of the IL-4R binding agent used. No difference is seen comparing the growth reducing effect of large molecules like total antibodies (IgG-format) to small molecules like scFvs, indicating that also small molecules have the capability to interfere with IL4 dependent signal transduction via binding to the IL-4R.

In a separate experiment a comparison of different epitope regions for binding agents are compared. It turns out that for small binding agents (e.g. anticalins, scFv, FN3 monobodies) the inhibition of IL-4 bioactivity is strongest if the epitope comprises at least one of the amino acids T178-P182 and R185. Small binding agents directed against other epitopes as outlined above likewise reduce bioactivity of IL-4, however small antigen binding agents directed against the epitopes mentioned here exhibit strongest biological effects.

### REFERENCES

http://www.ncbi.nlm.nih.gov/pubmed/7957181?ordinalpos=12&itool=EntrezSystem2. PE ntrez.Pubmed.Pubmed ResultsPanel.Pubmed RVDocSum
Aman et al., 1996, J Biol Chem., Nov 15;271(46):29265-70
Kabat et al., 1991, Sequences of Proteins of Immunological Interest, Fifth Edition. NIH Publication No. 91-3242.
Kelley et al., 2005, J Biol Chem., Jan 21;280(3):2205-12
Kindt et al., 2006, J ‘Immunology’, 6th edition, Palgrave MacMillan
Kondo et al., 1993, Science, Dec 17;262(5141):1874-7
Todaro M, 2008, Cell Cycle , 7, 309-313
MacFarlane et al., 2005, PNAS, Jun 6;103(23):8634-9
Russell et al., 1993, Science, Dec 17;262(5141):1880-3
Todaro et al., 2007 ,Cell Stem Cell , 4, 389-402
Vercelli D, Curr Opin Immunol 2006, 8, 733-737
Tony et al., 1994, Eur J Biochem., Oct 15;225(2):659-65
Van der Sloot et al., 2006, Cancer Res., Dec 15;65(24):11265-70

1. An antigen-binding agent directed against IL-4R that does not interfere with the binding of IL-4 to IL-4R for treatment and/or prevention of tumors, inflammatory and immunological disorders.

2. The antigen-binding agent of claim 1, wherein the antigen-binding agent is selected from a monoclonal antibody, a chimeric antibody, a partially or fully humanized antibody, a fully human antibody, a single chain antibody, a whole antibody, an Fab, an F(ab′)2 fragment, an Fd fragment, a disulfide-linked Fvs (sdFvs), an anti-idiotypic (anti-Id) antibody, an scFvs, a miniantibody, a fragment of an antibody, an affibodies, a trinectin, a monobody, a FN3 monobody, an anticalin or an antibody mimetic.

3. The antigen-binding agent of claim 2, wherein the antigen-binding agent
(i) is produced by the hybridoma cell line DSM ACC 2882; or an antibody or antibody fragment derived thereof; or
(ii) recognizes the same epitope on human IL-4 receptor as the antibody of (i).

4. The antigen-binding agent of claim 1, wherein the antigen-binding agent comprises at least one heavy chain variable region and at least one light chain variable region, wherein the amino acid sequences of the complementarity-determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and/or iv) a sequence derived by substituting 1, 2 or 3 amino acids of SEQ ID NOs: 1, 2, or 3; and/or the amino acid sequences of the complementary determining regions (CDRs) of the light chain are: i) SASQDINNYLN (SEQ ID NO:4); ii) YTSSLHS (SEQ ID NO:5); iii) QQFSNLPWT (SEQ ID NO:6); and/or iv) a sequence derived by substituting 1, 2 or 3 amino acids of SEQ ID NOs: 4, 5, or 6.

5-13. (canceled)

14. Use of an antigen-binding agent directed against IL-4R that does not interfere with the binding of IL-4 to IL-4R for manufacture of a medicament for treatment of cancer, inflammatory and immunological disorders.

15. The use of claim 14, wherein the antigen-binding agent is selected from a monoclonal antibody, a chimeric antibody, a partially or fully humanized antibody, a fully human antibody, a single chain antibody, a whole antibodies, an Fab, an F(ab′)2 fragment, an Fd fragment, a disulfide-linked Fvs (sdFvs), an anti-idiotypic (anti-Id) antibody, an scFvs, a miniantibody, a fragment of an antibody, an affibodies, a trinectin, a monobody, a FN3 monobody, an anticalin or an antibody mimetic.

16-31. (canceled)

32. A method for inhibiting the bioactivity of IL-4 comprising administering to an individual in need thereof an antigen-binding agent with binding affinity for IL-4R.

33. The method of claim 32, wherein the IL-4R binding of the antigen-binding agent does not interfere with the binding of IL4 to IL-4R.

34. The method of claim 32, wherein the antigen-binding agent is a monoclonal antibody, a chimeric antibody, a partially or fully humanized antibody, a fully human antibody, a single chain antibody, awhole antibody, an Fab, an F(ab′)₂ fragment, an Fd fragment, a disulfide-linked Fvs (sdFvs), an anti-idiotypic (anti-Id) antibody, an scFvs, a miniantibody, a fragment of an antibody, an affibody, a trinectin, a monobody, a FN3 monobody, an anticalin, or an antibody mimetic.

35. The method of claim 32, wherein the antigen-binding agent
(i). is produced by the hybridoma cell line DSM ACC 2882; or an antibody or antibody fragment derived therefrom; or
(ii) recognizes the same epitope on human IL-4 receptor as the antibody of (i).

36. The method of claim 32, wherein the antigen-binding agent comprises at least one heavy chain variable region and at least one light chain variable region, wherein the amino acid sequences of the complementarity-determining regions (CDRs) of the heavy chains are i) SGFTFNTNAMN (SEQ ID NO:1), ii) RIRSKSNNYATYYADSVKD (SEQ ID NO:2); iii) DRGWGAMDY (SEQ ID NO:3); and/or iv) a sequence derived by substituting 1, 2 or 3 amino acids of SEQ ID NOs: 1, 2, or 3; and/or the amino acid sequences of the complementary determining regions (CDRs) of the light chain are: i) SASQDINNYLN (SEQ ID NO:4); ii) YTSSLHS (SEQ ID NO:5); iii) QQFSNLPWT (SEQ ID NO:6); and/or iv) a sequence derived by substituting 1, 2, or 3 amino acids of SEQ ID NOs: 4, 5, or 6.

37. The method of claim 32, wherein the antigen-binding agent is directed against an epitope naturally present on IL-4R comprising one, two, three, four, five, or more amino acids located within a region selected from the group consisting of amino acids H87-L89, R173-Y175 or T178-P182, D102-A125, W104-A125, W111-A125, H120, W111, K112, K116, T211, Y179 and R185 of SEQ ID NO: 12.

38. The method of claim 32, wherein the antigen-binding agent with binding affinity for IL4R is a bi-specific antigen-binding agent having at least one further binding affinity.

39. The method of claim 38, wherein the further binding affinity is a binding affinity for a cytokine molecule or a cytokine receptor molecule.

40. The method of claim 39, wherein the cytokine molecule or the cytokine receptor molecule is IL4, IL5, IL6; IL10; IL13, IL10R; IL13R, common chain or CXCR4.

41. The method of claim 32, wherein the inhibiting of the bioactivity of IL-4 is performed in the course of treatment and/or prevention of cancer, inflammatory or immunological disorders.

42. The method of claim 41, wherein the tumor is at least partially resistant to apoptosis.

43. The method of claim 41, wherein the tumor is an epithelial cancer, particularly a solid tumor.

44. The method of claim 41, wherein the tumor is selected from the group consisting of thyroid carcinoma, breast carcinoma, lung carcinoma, prostate carcinoma, bladder carcinoma, colon carcinoma, gastric carcinoma, liver carcinoma, kidney carcinoma, glioblastome, and MRD.

45. The method of claim 41, wherein the tumor is colon carcinoma or pancreas carcinoma.

46. The method of claim 41, wherein the inflammatory disorder is asthma, arthritis, cystic fibrosis, a lung disorder, tuberculosis, or dermatitis.

47. The method of claim 41, further comprising administering at least one chemotherapeutic agent to the individual.

48. The method according to claim 47, wherein the chemotherapeutic agent is selected from the group consisting of antimetabolites, DNA-fragmenting agents, DNA-crosslinking agents, intercalating agents, protein synthesis inhibitors, topoisomerase I and II inhibitors, microtubule-directed agents, kinase inhibitors, hormones and hormones antagonists.

49. The method according to claim 47, wherein the chemotherapeutic agent is selected from the group consisting of taxanes, platinum compounds, doxorubicin, and etoposide.

50. The method of claim 41, further comprising administering at least one further cytokine antagonist antibody, to the individual.

51. The method of claim 41, further comprising administering at least one death pathway agonist to the individual.
